(19) United States
(12) Patent Application Publication
(10) Pub. No.: US 2026/0240685 A1
(43) Pub. Date: Aug. 20, 2026

(54) INSERTABLE EYE SHIELD

(71) Applicant: Laservision USA, LP, St. Paul, MN (US)

(72) Inventor: Andrew Barrows, Bradenton, FL (US)

(21) Appl. No.: 19/540,300

(22) Filed: Feb. 13, 2026

Related U.S. Application Data
(60) Provisional application No. 63/758,766, filed on Feb. 14, 2025.

(51) Int. Cl. A61F 9/04; A61F 9/02

(52) U.S. Cl. CPC A61F 9/045; A61F 9/025; A61F 9/029; A61F 9/04

(57) ABSTRACT

An eye shield for protecting an eye during a medical procedure is made from a silicone and aluminum mixture and has a base portion generally conforming to the eye and a handle portion extending upwardly therefrom. The mixture is flexible and also reflects high energy waves such as laser light.

### CROSS REFERENCE TO RELATED APPLICATION

This patent application claims the benefit of U.S. Provisional Application No. 63/758,766 titled INSERTABLE EYE SHIELD, filed Feb. 14, 2025, the content of which is expressly incorporated by reference herein in its entirety.

### FIELD OF THE DISCLOSURE

The invention relates to eye protection. More specifically, the invention relates to an eye shield to protect an eye during medical procedures.

### BACKGROUND

High energy waves are often used for medical procedures. For example, lasers may be used for ablation, cauterization, or the like.

### SUMMARY

Lasers may be used on the skin, for procedures such as hair removal, tattoo removal, blemish removal, wrinkle removal or the like. IPL (intense pulsed light) therapy is a non-ablative cosmetic treatment that uses high-intensity pulses of visible light.

An eye shield for protecting an eye during a medical procedure includes a base portion and a handle portion. The base portion has a dome shape with a curved interior surface generally conforming to the eye. The handle portion extends upwardly from a peak of the dome-shaped base portion. The handle portion and base portions are made from a silicone and aluminum mixture. In embodiments of the invention, the silicone and aluminum mixture is configured to reflect high energy waves. A set of eye shields includes at least 3 eye shields. The base portion of each of the eye shields in the set has a different diameter from one another. Each of the different diameters is between 16 and 24 mm.

### BRIEF DESCRIPTION OF THE DRAWINGS

FIG. 1 is a perspective view of a set of eye shields in accord with embodiments.

FIG. 2A is a side view of the set of eye shields of FIG. 1.

FIG. 2B is a top view of the set of eye shields of FIG. 1.

FIG. 2C is a bottom view of the set of eye shields of FIG. 1.

FIG. 3 is a cross-sectional view of the set of eye shields of FIG. 1.

FIG. 4 is an eye shield being placed on an eye of a patient in accord with embodiments.

FIG. 5 is an eye shield protecting an eye from a laser source in accord with embodiments.

FIG. 6 is packaging for an eye shield in accord with embodiments.

While embodiments of the disclosure are amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

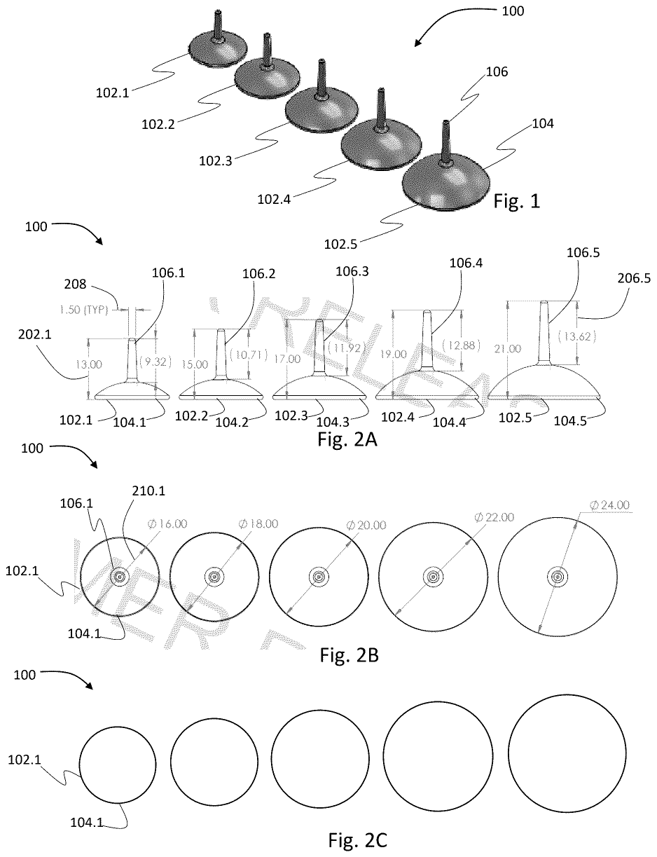

Referring to FIG. 1, a set of eye shields 100 is shown with eye shields 102 of varying sizes. Each eye shield 102 has a base portion 104 that generally conforms to a patient's eye and a handle portion 106. The set of eye shields 100 is shown arranged in order of size from a small eye shield 102.1 to a large eye shield 102.5. Providing a set of shields 100 with a plurality of different sized eye shields 102 allows a user to readily select an appropriately-sized eye shield 102 to correspond to a patient's eye. For example, a child may require a small eye shield 102.1, a teenager may require a medium eye shield 102.3, and an adult may require a large eye shield 102.5. Although the set of eye shields 100 is depicted in FIG. 1 as including 5 eye shields 102, alternate sets of eye shields 100 are not beyond the scope of this disclosure. In embodiments, a set of eye shields 100 may include between 1 and 10 eye shields 102. In embodiments, a set of eye shields may include 1, 2, 3, 4, or 5 eye shields 102. In embodiments, each eye shield 102 in a set 100 may all be the same size. In some embodiments, a set of eye shields 100 may include pairs of similarly sized eye shields 102. For example, a set of eye shields 100 may include two small eye shields 102.1 and two large eye shields 102.5. In other embodiments, a set of eye shields 102 may comprise or consist of a single pair of same-sized eye shields, one for each eye of a single patient.

Although a plurality of eye shields 102 may be provided as a set, it will be understood that a single eye shield 102 may be provided.

Referring to FIG. 2A, a side view of a set of eye shields 100 is depicted. The handle portion 106 facilitates placing the eye shield 102 on an eye, as well as facilitates removal of the eye shield 102 after completion of a procedure. The handle portion 106 presents a ready grip for an operator to grasp and maneuver the eye shield 102. In some embodiments, the handle portion 106 is unitary with the base portion 104. The handle portion 106 has a height 206 and extends generally upwards from a central point at the apex of the base portion 202. In some embodiments, the height 206 of the handle portion 106 is proportional to a size of the eye shield 102. For example, a small eye shield 102.1 may have a shorter handle portion 106.1 than a large eye shield 102.5. In embodiments, the height 206 of the handle portion 106 may range from 5 mm to 20 mm. In embodiments, the height 206 of the handle portion 106 may range from about 9 mm to about 14 mm. In embodiments, the height 206 of each handle portion 106 in a set of eye shields 100 may be about 1 to 1.5 mm larger or smaller than the height of the handle portion of the next closest in size eye shield. Size may also be based on a user's ability to grasp the handle. In embodiments, a handle portion 106 has a diameter 208 near a top end of about 1.5 mm. The handle portion 106 may taper slightly such that the handle portion 204 may be slightly wider where it meets the base portion 104 than at the opposite, top end. In embodiments, there is little variance between a diameter of handle portion 106 between different sized eye shields 102 within a set 100.

In embodiments, a height 202 of the eye shield 102, as measured from a bottom edge of the base portion 104 to the top of the handle portion 106 may range from about 13 mm to about 21 mm. In embodiments, the height 202 of each eye shield 102 in a set of eye shields 100 may be about 2 mm larger or smaller than the height of the next closest in size eye shield. In embodiments, a set of eye shields 100 may include 5 eye shields 102, each eye shield 102 having a height of 13 mm, 15 mm, 17 mm, 19 mm, and 21 mm respectively. Referring to FIG. 2B, a top view of a set of eye shields 100 is shown, and FIG. 2C illustrates a bottom view of the set 100. As shown, in the top and bottom views, base portions 104 are generally circular. Each eye shield 102 has a diameter 210. A diameter 210 of an eye shield may range from about 16 mm to about 24 mm. In embodiments, the diameter 210 of each eye shield 102 in a set of eye shields 100 may be about 2 mm larger or smaller than the diameter of the next closest in size eye shield. In embodiments, a set of eye shields 100 may include 5 eye shields 102, each eye shield 102 having a diameter of 16 mm, 18 mm, 20 mm, 22 mm, and 24 mm respectively.

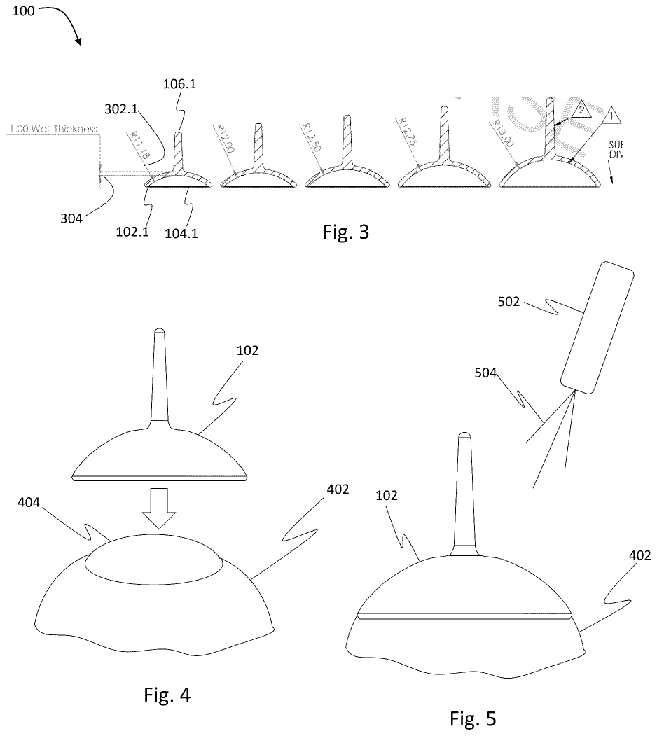

Referring to FIG. 3, a cross-sectional side view of a set of eye shields 100 is depicted. The lower portion 104 of each eye shield 102 has a concave interior surface 302 that generally conforms to a patient's eye. A radius of curvature 304 of the interior surface 302 may range from about 11.18 degrees to about 13 degrees, and in other embodiments from about 10 degrees to about 15 degrees. In embodiments, the radius of curvature 304 of each eye shield 102 in a set of eye shields 100 may vary between about 0.25 and about 0.75 degrees as compared to the next closest in size eye shield. In embodiments, a set of eye shields 100 may include 5 eye shields 102, each eye shield 102 having a radius of curvature of about 11.18, 12, 12.5, 12.75, and 13 degrees respectively.

In embodiments, each eye shield 102 has a wall thickness 304 of about 1 mm. Each eye shield 102 is molded as a single piece from the same material. Each eye piece is formed from a flexible mixture of silicone and aluminum. In embodiments, the flexible mixture is about 90% silicone and about 10% aluminum. In embodiments, the flexible mixture is between 80 to 95% silicone, with the remaining percentage of the flexible mixture being aluminum. The silicone portion of the material provides flexible characteristics to the eye shield making allowing it to conform to the eye and making the shield generally more comfortable to wear than a metallic shield. The aluminum blocks/reflects light, such as high energy light used during medical procedures that would otherwise damage the eye. In an embodiment, to form the shield, the aluminum is melted and mixed with the silicone forming a single molten material. This process prevents deposits of aluminum fibers or shards that might otherwise scratch or damage the eye.

Referring to FIG. 4, the eye shield 102 may be placed on a patient's eye 402, such as before a medical procedure. In an embodiment, when the shield 102 is fully in place, as illustrated in FIG. 5, the cornea 404 is completely covered by the shield 102. Accordingly, the eye shield 102 protects not only the surface of the eye 402, but it also protects interior structures of the eye, such as the retina, that are particularly sensitive to, and easily damaged by, exposure to high energy light waves. Eyelids and other anatomical features may surround the eye shield 102 (not shown), thereby providing near-complete protection in cooperation with an eye shield 102. Referring to FIG. 5, during medical procedures, a high-energy light source 502 may direct high-energy light waves 504 towards the eye. For example, lasers may be used to treat skin on a patient directly adjacent to the eye. The eye shield 102 may protect the eye 402 and/or portions of the eye such as the cornea 404 from the energy waves 504.

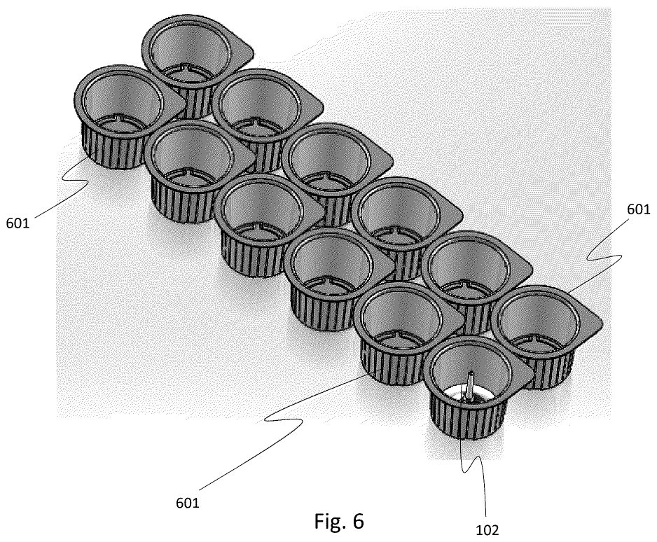

Referring to FIG. 6, a plurality of packages 601 for receiving the eye shield 102 is shown, each package 601 generally conforming to a single eye shield 102. The packaging 601 has a diameter and a height sufficient to contain an eye shield 102. In embodiments, the eye shield 102 may be disinfected before being inserted into the package. In embodiments, the packaging 601 is disinfected before receiving an eye shield 102. In embodiments, the combination of the packaging 601 and eye shield 102 are disinfected. Accordingly, the packaging 601 may be opened and an eye shield 102 placed directly on a patient's eye without further handling required by the operator. The packaging 601 may be sealed (not shown) such as by shrink wrap and/or a lid, such as those understood in the art. Such a lid may be, for example, a foil or similar lid and may be secured with heat adhesive and/or application of heat.

Each feature disclosed in this specification (including references incorporated by reference, any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any incorporated by reference references, any accompanying claims, abstract and drawings), or to any novel one, or any novel combination, of the steps of any method or process so disclosed. The above references in all sections of this application are herein incorporated by references in their entirety for all purposes.

Although specific examples have been illustrated and described herein, it will be appreciated by those of ordinary skill in the art that any arrangement calculated to achieve the same purpose could be substituted for the specific examples shown. This application is intended to cover adaptations or variations of the present subject matter. Therefore, it is intended that the invention be defined by the attached claims and their legal equivalents, as well as the following illustrative aspects. The above described aspects embodiments of the invention are merely descriptive of its principles and are not to be considered limiting. Further modifications of the invention herein disclosed will occur to those skilled in the respective arts and all such modifications are deemed to be within the scope of the invention.

What is claimed is:

1. An eye shield for protecting an eye during a medical procedure comprising:
a base portion having a dome shape with a curved interior surface generally conforming to the eye; and
a handle portion extending upwardly from a peak of the dome shaped base portion,
wherein the handle portion and base portions are made from a silicone and aluminum mixture.

2. The eye shield of claim 1, wherein the base portion is flexible.

3. The eye shield of claim 1, wherein the silicone and aluminum mixture comprises about 90% silicone and about 10% aluminum.

4. The eye shield of claim 1, wherein the silicone and aluminum mixture comprises about 85%-90% silicone and about 10%-15% aluminum.

5. The eye shield of claim 1, wherein the silicon and aluminum mixture forms a single material formed from melted aluminum and silicon.

6. The eye shield of claim 1, wherein the silicone and aluminum mixture is configured to reflect high-energy light.

7. The eye shield of claim 1, wherein the base portion has a wall thickness of about 1mm.

8. The eye shield of claim 1, wherein the base portion and the handle portion form a unitary structure.

9. The eye shield of claim 1, wherein the handle is tapered such that an end of the handle proximal to the base portion has a larger diameter than an end of the handle distal to the base portion.

10. The eye shield of claim 1, wherein a height of the handle ranges from 5 mm to 20 mm.

11. The eye shield of claim 1, wherein a height of the handle ranges from 9 mm to 14 mm.

12. The eye shield of claim 1, wherein the base portion is generally circular.

13. The eye shield of claim 12, wherein a radius of curvature of a concave inside surface of the base portion is configured to conform to a patient's eye, having a radius of curvature ranging from about 11.2 degrees to about 13 degrees.

14. A set of eye shields, comprising:
at least three of the eye shields of claim 1, wherein the base portion of each of the eye shields has a different diameter from one another.

15. The set of eye shields of claim 14, wherein each of the different diameters is between 16 and 24 mm.

16. A set of eye shields, comprising:
a plurality of eye shields, each eye shield for protecting an eye during a medical procedure and each eye shield including:
a generally circular base portion having a dome shape with a curved interior surface generally conforming to the eye, the curved interior surface having a radius of curvature in a range of from about 11 degrees to about 13 degrees; and
a tapered handle portion extending upwardly from a peak of the dome-shaped base portion, a height of the tapered handle portion being in a range of about 5 mm to about 20 mm;
wherein the handle portion and base portions are made from a silicone and aluminum mixture that comprises a majority of silicone material;
wherein the plurality of eye shields includes a first eye shield with a first base having a first base diameter and first inside radius of curvature, and a first handle having a first handle height and a second eye shield with a second base having a second base diameter and second inside radius of curvature, and a second handle having a second handle height; and
wherein the first base diameter is greater than the second base diameter or the first inside radius of curvature is greater than the second inside radius of curvature or the first handle height is greater than the second handle height.

17. The set of eye shields of claim 16, wherein the first base diameter is greater than the second base diameter and the first inside radius of curvature is greater than the second inside radius of curvature and the first handle height is greater than the second handle height.

18. The set of eye shields of claim 17, wherein the silicone and aluminum mixture comprises about 90% silicone and about 10% aluminum.

19. The set of eye shields of claim 16, wherein a base diameter of each of the plurality of eye shields in between 16 and 24 mm.

20. An eye shield for protecting an eye during a medical procedure comprising:
a generally circular base portion having a dome shape with a curved interior surface generally conforming to the eye, the curved interior surface having a radius of curvature in a range of from about 11 degrees to about 13 degrees; and
a tapered handle portion extending upwardly from a peak of the dome-shaped base portion, a height of the tapered handle portion being in a range of about 5 mm to about 20 mm;
wherein the handle portion and base portions are made from a silicone and aluminum mixture that comprises about 90% silicone and about 10% aluminum.
